(19) United States
(12) Patent Application Publication
(10) Pub. No.: US 2007/0142724 A1
(43) Pub. Date: Jun. 21, 2007

(54) Medium and method for measuring the efficacy of a tumour therapy

(75) Inventors: Alexandra Babarina, Munchen (DE); Dirk Sebastian Waldenmaier, Munchen (DE); Frank Christian Kischkel, Lubbecke (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Cell Control Diagnotics GmbH & Co., 82152 Planegg-Martinsried (DE)

(21) Appl. No.: 10/534,268

(22) PCT Filed: Nov. 6, 2003
(86) PCT No.: PCT/EP03/12428

§ 371 Date: Apr. 11, 2006

(30) Foreign Application Priority Data
Nov. 7, 2002 (DE) 102 51 879.2

(51) Int. Cl. A61B 6/00

(52) U.S. Cl. 600/431

(57) ABSTRACT

According to the invention, a measurement of the efficacy of a tumour therapy in single cell suspensions of tumour cells, by determination of the acid formation in a medium in the presence and the absence of a cytotoxic substance, is carried out by performing the measurement in a medium comprising 0.1 to 1 mM of a pH 7.0 to 7.4 buffer, 2 to 10 g/l glucose, 2 to 5 mM glutamine as the carbon source and 5 to 20 vol. % fetal calf serum.

The invention relates to a medium for measuring the efficacy of a tumor therapy on single cell suspensions and to a method for measuring the efficacy of a tumor therapy by using this medium.

In order to test the efficacy of tumor therapies it would be desirable to be able to determine on an isolated tumor cell the effect of a tumor therapy on the viability of this cell. Such a method would make it possible to establish beforehand whether a particular therapy is effective or not for a patient's specific tumor. For this purpose it would he necessary for parts of the tumor to be removed and be converted by enzymatic digestion into a single cell suspension, and then to determine the vitality of the single cells thus obtained under the influence of the therapy, i.e. typically of the chemotherapeutic agent. Comparison of the metabolic activity of untreated tumor cells with the metabolic activity of therapeutically treated tumor cells then allows direct determination of the efficacy of the therapy on the basis of the reduced metabolic activity of the treated cell compared with the untreated tumor cell. In this way it would be possible to determine within the shortest possible time which therapy is suitable or less suitable for the treatment of the patient with a particular tumor.

A crucial problem for such a method is, however, adjusting the conditions in the cell suspension so that the isolated tumor cells can display maximum metabolic activity. Only in this way is it possible to measure a reduction in the metabolic activity with sufficient accuracy. However, single cell suspensions of tumor cells prepared from a compact tumor tissue by enzymatic digestion display extremely low metabolic activities in known media, so that it is possible only with great difficulty or not at all to determine a further reduction in the metabolic activity with sufficient accuracy.

The invention is based on the object of providing a cell culture medium which to confer a maximum metabolic activity and lifespan on single cell suspensions of tumors prepared in this way, and of designing a method for assessing a tumor therapy using this medium.

This object is achieved according to the invention by a medium for measuring the efficacy of a tumor therapy on single cell suspensions comprising the essential amino acids, vitamins, salts and carbon donors, characterized in that the medium comprises from 0.1 to 1 mM buffer of pH 7.0 to 7.4, 2 to 10 g/l glucose and 2 to 5 mM glutamine as carbon sources and 5 to 20% fetal calf serum.

The inventive medium differs from known media by a very low buffer capacity, a higher content of glucose, glutamine and fetal calf serum. The medium preferably does not contain carbon sources other than glucose and glutamine and other buffers.

The composition of the inventive medium depends to a certain extent on the metabolic properties of the cells present in the investigated tumor. Adjustment of the variables in the inventive medium aims at adjusting the conditions of glycolysis for the tumor cells, because in this case a large change in pH is to be expected. It is at the same time intended that normal cells, which are unavoidably likewise present in the suspension, are kept where possible in aerobic metabolism and thus exert a negligibly small effect on the pH.

Since tumor cells consume more glucose than many non-tumor cells and, on the other hand, the aim in tumor cells is to achieve maximal glycolysis, when the proton formation and thus the acidification of the medium per ATP molecule produced is at a maximum, the amount of glucose in the inventive medium is preferably more than 2 g/l, particularly preferably from 4 to 6 g/l, glucose. Other carbon sources except the glutamine which is present as essential ingredient of the medium should preferably not be present. Typical examples of such unwanted carbon sources are pyruvate, lipids and similar known carbon sources in nutrient media. Lipids in particular proved to be deleterious in combination with glutamine because the initial activity of all tumor cells is greatly reduced thereby.

The best results in terms of increasing the activity of tumor cells with, at the same time, a minimal influence of non-tumor cells on the change in pH was therefore afforded a content of 5 g/l glucose, 2 mM glutamine and 10% by volume fetal calf serum, it being possible for each of these three values to vary by ±10%.

The inventive medium can be prepared by assembly from the components. For example, conventional vitamin mixtures and protein hydrolyzates can be assembled as source of the essential amino acids with the abovementioned essential amounts of glucose, glutamine, buffer and fetal calf serum. It is also possible alternatively to start from known media and supplement their composition by adding the important amounts of essential ingredients as explained above. A particularly suitable initial basis for this is the RUN medium, The medium may additionally comprise antibiotics.

The invention further relates to a method for measuring the efficacy of a tumor therapy on single cell suspensions of tumor cells by comparing the acid formation in a medium on therapeutically treated and untreated tumor cells, which is characterized in that the measurement is carried out in a medium as described above. The method is very suitable for being carried out with use of commercially available devices such as, for example, the Cytosensor® microphysiometer from Molecular Devices Corp. Sunnyvale, Calif., USA, but is not restricted thereto.

The latter device and its use for analyzing cell membrane-bound receptors is disclosed in Biosensors & Bioelectronics 15 (2000) 149-158, with measurement of the pH changes caused by the physiological effects of such receptors being carried out as a measure of the metabolic affects.

The inventive method for measuring the efficacy of a tumor therapy is described below for example with use of the Cytosensor® microphysiometer.

To carry out the inventive method with use of the inventive medium, a sample of the tumor to be investigated is removed and converted into a single cell suspension by enzymatic digestion in a known manner. The cells are removed from the digestion medium and then their pH curve in the inventive medium is recorded. A typical medium according to the invention has, for example, the composition to be found in Table 1 below.

TABLE 1

| Concentration | | |
|---|---|---|
| mg/l | | Substance |
| 4.45 | | alanine, L- |
| 147.5 | | arginine L-, HCl |
| 7.5 | | asparagine, L-, H₂O |
| 6.65 | | aspartic acid, L- |
| 24 | | cystine, L- |
| 17.56 | | cysteine, L-, HCl, H₂O |
| 7.35 | | glutamic acid, L- |
| 18.75 | | glycine |
| 31.48 | | histidine, L-, HCl H₂O |
| 20 | | hydroxyproline, L-4- |
| 54.45 | | isoleucine, L- |
| 59.05 | | leucine, L- |
| 91.25 | | lysine, L-HCl |
| 17.24 | | methionine, L- |
| 35.48 | | phenylalanine, L- |
| 17.25 | | proline, L- |
| 26.25 | | serine, L- |
| 53.45 | | threonine, L- |
| 9.02 | | tryptophan, L- |
| 38.7 | | tyrosine, L- |
| 52.85 | | valine, L- |
| 2000 | | D + glucose |
| 12.6 | | inositol, myo-, inositol, meso- |
| 0.00365 | | biotin, D- |
| 2.24 | | calcium D-pantothenate |
| 8.98 | | choline chloride |
| 1 | | glutathione, L-, reduced |
| 2.02 | | nicotinamide |
| 2.031 | | pyridoxine, HCl |
| 2.17 | | thiamine chloride, thiamine HCl |
| 0.01 | | tocopherol succinate DL-alpha |
| 0.1 | | vitamin A acetate |
| 0.68 | | vitamin B 12 (cyanocobalamin) |
| 2 | | vitamin C (ascorbic acid) |
| 0.1 | | vitamin D2 |
| 8.I | | phenol red |
| 154 | | calcium chloride 2H₂O |
| 0.05 | | iron(III) nitrate, 9H₂O |
| 0.417 | | iron(II) sulfate, 7H₂O, analytical grade |
| 311.8 | | potassium chloride |
| 0.00125 | | copper(II) sulfate, 5H₂O |
| 100 | | magnesium sulfate, 7H₂O |
| 61 | | magnesium chloride, 6H₂O |
| 6999.5 | | sodium chloride |
| 14.2 | | disodium hydrogen phosphate (anhyd.) |
| 0.43 | | zinc sulfate, 7H₂O |
| 2 | mM | glutamine |
| 10 | vol % | fetal calf serum |
| 100 | units/ml | penicillin |
| 100 | μg/ml | streptomycin |
| 100 | μg/ml | kanamycin |
| 2.5 | μg/ml | Fungizone |

For this purpose, the single cells obtained in this way are then preferably immobilized on a pH electrode. The immobilization can take place by suitable substances as usual in cell culture. Agarose has proved to be very suitable, being able to immobilize the isolated single cells on the electrode surface, but other immobilizing agents used in cell culture can also be used.

The instrument used has eight channels in which the pH can be determined simultaneously in flow cells. A pH electrode on which the immobilized cell suspension is present is disposed in each channel. The inventive medium is then pumped over this immobilized cell suspension by a pump for a suitable period and, at the same time, the pH measurement is started. In a typical case, a measurement cycle lasts 120 seconds. For 90 seconds of this only medium is pumped in, and a pH is determined every second. Then the change in pH is measured for 30 seconds. This procedure is continued over a lengthy period, usually 14 to 24 hours, so that the final result is a pH curve which is directly equivalent to the metabolic activity of the immobilized cells.

As mentioned above, there are eight parallel channels in the instrument indicated above. To determine the efficacy of a tumor therapy it is expedient to pump only inventive medium in one channel to determine a basic plot, and various antitumor agents (cytostatics) can be determined simultaneously in the other channels. Since the cytostatics, if they display an efficacy in relation to tie tumor cells used herein, lead to a reduction in the metabolic activity, a pH curve obtained over the course of time results in a smaller pH change as a result of the reduced metabolic activity due to the action of the cytostatics. It is possible from comparison of the curve inclinations which are then obtained for the curves measured in the presence of cytostatics compared with the cytostatic-free plot to determine directly the activity of the compounds having cytostatic activity investigated in each case. It is also possible in the same way not to investigate different compounds having cytostatic or cytotoxic activity in the different channels, but to investigate different concentrations of a particular cystostatic for their efficacy. The latter embodiment has the advantage that any effects of the investigated cytostatic on the pH can be compensated by adapting the medium within the inventive limits, in particular in relation to the buffer capacity. The buffer capacity as such is kept as low as possible but may not be below 0.1 mM. The amount of buffer is preferably set as close as possible to this lower limit. However, the amount of buffer can be increased up to a maximum of 1 mM for cytostatics which may themselves affect the pH.

The invention makes extracorporeal measurement of the efficacy of a tumor therapy in a patient possible within a very short time and then appropriate adjustment of the treatment on the basis of the measurement results. Thus, not only is a crucial saving in time in the treatment of the patient made possible, but the risk of unwanted side effects of the therapy without a corresponding efficacy in relation to the tumor itself is also reduced or entirely prevented. This constitutes a considerable advance in tumor therapy.

1. A medium for measuring the efficacy of a tumor therapy on single cell suspensions, comprising the essential amino acids, vitamins, salts and carbon donors, characterized in that the medium comprises from 0.1 to 1 mM buffer of pH 7.0 to 7.4, 2 to 10 g/l glucose, 2 to 5 mM glutamine as carbon source and 5 to 20% by volume fetal calf serum.

2. The medium as claimed in claim 1, characterized in that it comprises phosphate buffer as buffer.

3. The medium as claimed in claim 1 or 2, characterized in that it comprises from 8 to 12% by volume fetal calf serum.

4. The medium as claimed in any of claims 1 to 3, characterized in that the medium comprises 5 g/l glucose, 2 mM glutamine and 10% by volume fetal calf serum, it being possible for each of these values to vary by 10%.

5. The medium as claimed in any of claims 1 to 4, characterized in that the medium comprises 5 g/l glucose, 2 mM glutamine and 10% by volume fetal calf serum, it being possible for each of these values to vary by 10%.

6. A method for measuring the efficacy of a tumor therapy on single cell suspensions of tumor cells by determining the acid formation in a medium in the presence and in the absence of a substance having cytostatic or cytotoxic activity, characterized in that the measurement is carried out in a median as claimed in any of claims 1 to 4.

7. The method as claimed in claim 6, characterized in that the measurement is carried out by means of a pH electrode on which the single cell suspension is immobilized, with use of a flow cell through which the inventive medium flows.

8. The method as claimed in claim 6 or 7, characterized in that the medium is pumped through the flow cell until a constant pH has been set up, and the change in the pH with the medium stationary is then measured by measuring at short intervals, the medium is thereafter removed from the measurement cell, and the measurement cycle is started from the beginning again until the pH change has been determined over a lengthy period.

9. The method as claimed in claim 8, characterized in that the medium is supplied and measured for 1½ to 2½ minutes and then the procedure is repeated with fresh medium for 14 to 24 hours.

10. The method as claimed in claim 9, characterized in that the method is carried out in a multichannel instrument, with one channel being charged by a medium without cytostatic and the other channels being charged with the same medium which comprises the cytostatic to be investigated.
